(19) United States
(12) Patent Application Publication
(10) Pub. No.: US 2007/0048339 A1
(43) Pub. Date: Mar. 1, 2007

(54) Structured materials

(76) Inventors: Lewis Michael Popplewell, Morganville, NJ (US); Lulu Henson, Plainsboro, NJ (US)

Correspondence Address: INTERNATIONAL FLAVORS & FRAGRANCES INC., 521 WEST 57TH ST, NEW YORK, NY, 10019, US

(21) Appl. No.: 11/216,695

(22) Filed: Aug. 31, 2005

(51) Int. Cl. A61K 8/02

(52) U.S. Cl. 424/401; 510/300

(57) ABSTRACT

The present invention relates to a method for preparing a structured material comprising: providing a hydrophilic particle such as hydrophilic silicas, silicates, dextrins, starches, minerals, sodium bicarbonate, clays, sugars, polyols, dried flavors/fragrances, hydrocolloids, proteins, celluloses, methylcellulose, hydroxypropyl methyl cellulose, flours of wheat, corn, potato, and rice, dried milk and dairy powders, spice, herb, and vegetable powders, meat powders, salts, and acids; providing a hydrophobic active ingredient selected from the group consisting of flavors, fragrances, pharmaceutical and nutritional agents, and/or an appropriate hydrophobic carrier such as triglyceride oils, triglyceride fats and mineral oils; providing a polyhydric material such as glycerine, propylene glycol, dipropylene glycol, polyethylene glycol, aqueous solutions of sorbitol, isomalt, lactitol, maltitol, aqueous solutions of sugars, starches, acids, polyethylene glycols, dextrins, and water soluble or swellable compounds, or water; and admixing said hydrophilic particle, said active ingredient and said polyhydric material to form said structured material. The above method of the present invention will be apparent by reading the following specification.

### FIELD OF THE INVENTION

The present invention relates to preparation of stable structured materials and use of these structured materials in the flavor, fragrance, and pharmaceutical industries.

### BACKGROUND OF THE INVENTION

It is well known that liquid active ingredients such as flavors, fragrances, therapeutic or diagnostic agents and oils may be absorbed onto a variety of hydrophobic and hydrophilic solid particles in order to render them easier to handle and distribute. Often, fine particles with a large externally-available porosity are used due to their ability to carry high levels of these active ingredients while still retaining good flow properties. The porosity provides a large available surface area for absorption of the liquid film. Materials of this type include selected silicon dioxides, such as Syloid 244 by Grace Davison, tapioca dextrins such as N-Zorbit™ by National Starch and polypropylene, such as Accurel by Membrana GmbH. From the preceding examples, it is recognized that the surface of the particle may be either hydrophilic or hydrophobic, and that selection of the surface type will be dependent on the specific application.

The level of the absorbed active ingredient needs to be kept below a certain level in order to maintain the particle-active ingredient mixture as a flowable powder or granular product. This level is specific to each particle-active ingredient combination, and is normally determined experimentally. As the level of active ingredient is increased, the mixture becomes agglomerated and sticky. If the level is further increased, a paste, and finally a liquid suspension results. These suspensions are often useful, as they will have an increased viscosity in comparison to the original liquid which can make handling easier in specific situations.

Regardless of the type and level of particles used, it is difficult to create a stable solid structure that contains a high level of the liquid active ingredient. This is apparently due to the fact that while the particles can create a high apparent viscosity in a liquid, there is essentially no direct inter-particle interaction. Furthermore, these simple dispersions of particles in liquid materials do not help significantly in controlling the delivery of hydrophobic active ingredients such as flavors and fragrances, as they are not sufficiently stable in the aqueous application systems most often encountered.

Accordingly, a need exists to develop stable structures that contain high levels of active ingredient and that allow a controlled method of delivery of the active ingredient.

### SUMMARY OF THE INVENTION

The present invention relates to a method for preparing a structured material comprising:

providing a hydrophilic particle such as hydrophilic silicas, silicates, dextrins, starches, minerals, sodium bicarbonate, clays, sugars, polyols, dried flavors/fragrances, hydrocolloids, proteins, celluloses, methylcellulose, hydroxypropyl methyl cellulose, flours of wheat, corn, potato, and rice, dried milk and dairy powders, spice, herb, and vegetable powders, meat powders, salts, and acids;

providing a hydrophobic active ingredient selected from the group consisting of flavors, fragrances, pharmaceutical and nutritional agents, and/or an appropriate hydrophobic carrier such as triglyceride oils, triglyceride fats and mineral oils;

providing a polyhydric material such as glycerine, propylene glycol, dipropylene glycol, polyethylene glycol, aqueous solutions of sorbitol, isomalt, lactitol, maltitol, aqueous solutions of sugars, starches, acids, polyethylene glycols, dextrins, and water soluble or swellable compounds, or water; and

admixing said hydrophilic particle, said active ingredient and said polyhydric material to form said structured material.

The above method of the present invention will be apparent by reading the following specification.

### DETAILED DESCRIPTION OF THE INVENTION

It has been unexpectedly found that active ingredients such as flavors, fragrances, pharmaceutical, therapeutic and diagnostic agents may be effectively structured into stable solids, or viscous liquids, using hydrophilic particles and a small amount of polyhydric alcohol or like materials. While the exact mechanism has not been proven, it is thought that the polyhydric alcohol creates an association between the particles, thus resulting in a more stable structure than what can be obtained with particles alone. This allows the structures formed to be used effectively in delivering active ingredients. Specifically, these structures have been found useful in delivering flavors and fragrances in basic two ways:

a) The product is a structured liquid flavor or fragrance. These structures have been found to have stability in aqueous applications such as toothpaste; and

b) The product is a structured fat or oil, which contains dried particles of flavor or fragrance. These structures provide improved hydrophobic protection to the flavor or fragrance in a variety of aqueous applications.

The above approaches may be combined by using dried flavor/fragrance inside a structured liquid flavor or fragrance product or diluting a liquid flavor or fragrance using a fat or oil.

The concept of the present invention is clearly illustrated by the experiment described in the Example 3 below. As indicated in this example, an addition of about 5% of polyhydric alcohol glycerine approximately doubles the viscosity of the structured material. In this case the viscosity measured using a Haake AR 2000 Rheometer and a shear rate sweep from about 10,000 to about 140,000 Pa·s at about 25° C. The viscosity of the same material under the same conditions but without the polyhydric alcohol is in the range of from about 5,000 to about 55,000. The additional portion of the polyhydric alcohol increases the viscosity of the structured material. As further indicated in the same example, an addition of about 8% of the polyhydric alcohol glycerine to a sample containing a higher level of hydrophilic particles also greatly increases the viscosity of the structured material. In this case the viscosity measured using a Haake AR 2000 Rheometer and a shear rate sweep is from about 80,000 to about 1,000,000 Pa·s at about 25° C.

The structured materials of the present invention are formulated as follows:
Hydrophobic Active Ingredient: 50-90%, preferably 55-75%, most preferably 60-70% by weight of the structured material;
Hydrophilic Particles: 5-70%, preferably 10-50%, most preferably 20-30% by weight of the structured material; and
Liquid polyhydric material: 1-20%, preferably 10-15% by weight of the structured material.

As used herein the term structured material is understood to mean a material exhibiting increased viscosity, resistance to deformation, or the properties of a.solid; liquid polyhydric material is any material with more than one —OH group, water, or an aqueous solution of a soluble material; hydrophobic material is any material that tends not to dissolve or mix with water.

The active ingredient provided by the present invention consists largely of hydrophobic materials, although it may contain materials with some aqueous solubility, or materials with high water solubility in limited amounts. It may be any suitable agent including therapeutic and diagnostic agents, flavors, fragrances, triglyceride oils/fats, mineral oils and combinations thereof. Emulsifiers/surfactants such as mono-glycerides, di-glycerides, and polysorbates may form up to 30% of the structured material as well. The material must be a fluid during mixing.

A hydrophobic carrier solvent such as triglyceride oil may also solubilize the flavor or fragrance hydrophobic active ingredient and/or serve as the base for the hydrophobic particle. The amount of flavor/fragrance that is dissolved in the solvent may vary depending on the flavor/fragrance impact desired in the product.

A list of suitable fragrances is provided in U.S. Pat. No. 4,534,891. Another source of suitable fragrances is found in Perfumes, Cosmetics and Soaps, Second Edition, edited by W. A. Poucher, 1959. Among the fragrances provided in this treatise are acacia, cassie, chypre, cyclamen, fern, gardenia, hawthorn, heliotrope, honeysuckle, hyacinth, jasmine, lilac, lily, magnolia, mimosa, narcissus, freshly-cut hay, orange blossom, orchid, reseda, sweet pea, trefle, tuberose, vanilla, violet, wallflower, and the like.

Conventional flavoring materials useful in flavoring products include saturated fatty acids, unsaturated fatty acids and amino acids; alcohols including primary and secondary alcohols, esters, carbonyl compounds including ketones, other than the dienalkylamides of our invention and aldehydes; lactones; other cyclic organic materials including benzene derivatives, acyclic compounds, heterocyclics such as furans, pyridines, pyrazines and the like; sulfur-containing compounds including thiols, sulfides, disulfides and the like; proteins; lipids, carbohydrates; so-called flavor potentiators such as monosodium glutamate; magnesium glutamate, calcium glutamate, guanylates and inosinates; natural flavoring materials such as hydrolyzates, cocoa, vanilla and caramel; essential oils and extracts such as anise oil, clove oil and the like and artificial flavoring materials such as vanillin, ethyl vanillin and the like. High intensity sweetners such as aspartame and saccharin may also be used. Some of these flavoring materials may exist as solid particles.

Specific flavor adjuvants include but are not limited to the following: anise oil; ethyl-2-methyl butyrate; vanillin; cis-3-heptenol; cis-3-hexenol; trans-2-heptenal; butyl valerate; 2,3-diethyl pyrazine; methyl cyclo-pentenolone; benzaldehyde; valerian oil; 3,4-dimethoxy-phenol; amyl acetate; amyl cinnamate; γ-butyryl lactone; furfural; trimethyl pyrazine; phenyl acetic acid; isovaleraldehyde; ethyl maltol; ethyl vanillin; ethyl valerate; ethyl butyrate; cocoa extract; coffee extract; peppermint oil; spearmint oil; clove oil; anethol; cardamom oil; wintergreen oil; cinnamic aldehyde; ethyl-2-methyl valerate; γ-hexenyl lactone; 2,4-decadienal; 2,4-heptadienal; methyl thiazole alcohol (4-methyl-5-β-hydroxyethyl thiazole); 2-methyl butanethiol; 4-mercapto-2-butanone; 3-mercapto-2-pentanone; 1-mercapto-2-propane; benzaldehyde; furfural; furfuryl alcohol; 2-mercapto propionic acid; alkyl pyrazine; methyl pyrazine; 2-ethyl-3-methyl pyrazine; tetramethyl pyrazine; polysulfides; dipropyl disulfide; methyl benzyl disulfide; alkyl thiophene; 2,3-dimethyl thiophene; 5-methyl furfural; acetyl furan; 2,4-decadienal; guiacol; phenyl acetaldehyde; β-decalactone; d-limonene; acetoin; amyl acetate; maltol; ethyl butyrate; levulinic acid; piperonal; ethyl acetate; n-octanal; n-pentanal; n-hexanal; diacetyl; monosodium glutamate; mono-potassium glutamate; sulfur-containing amino acids, e.g., cysteine; hydrolyzed vegetable protein; 2-methylfuran-3-thiol; 2-methyldihydrofuran-3-thiol; 2,5-dimethylfuran-3-thiol; hydrolyzed fish protein; tetramethyl pyrazine; propylpropenyl disulfide; propylpropenyl trisulfide; diallyl disulfide; diallyl trisulfide; dipropenyl disulfide; dipropenyl trisulfide; 4-methyl-2-[(methyl-thio)-ethyl]-1,3-dithiolane; 4,5-dimethyl-2-(methylthiomethyl)-1,3-dithiolne; and 4-methyl-2-(methylthiomethyl)-1,3-dithiolane. These and other flavor ingredients are provided in U.S. Pat. Nos. 6,110,520 and 6,333,180.

It should be noted that flavor or fragrance materials and blends thereof will perform differently in the invention depending on their specific physical-chemical characteristics.

Examples of the appropriate therapeutic agents include hypnotics, sedatives, antiepileptics, awakening agents, psychoneurotropic agents, neuromuscular blocking agents, antispasmodic agents, antihistaminics, antiallergics, cardiotonics, antiarrhythmics, diuretics, hypotensives, vasopressors, antitussive expectorants, thyroid hormones, sexual hormones, antidiabetics, antitumor agents, antibiotics and chemotherapeutics, and narcotics.

Examples of diagnostic agents include, but are not limited to synthetic inorganic and organic compounds, proteins, peptides, polypeptides, polysaccharides and other sugars, lipids, and DNA and RNA nucleic acid sequences having diagnostic activities.

Examples of hydrophilic particles include hydrophilic silicas, silicates, dextrins, starches, minerals, sodium bicarbonate, acids, salts, clays, sugars, polyols, spray-dried flavors/fragrances, hydrocolloids, proteins, celluloses, flours of wheat, corn, potato, and rice, dried milk and dairy powders, spice, herb, and vegetable powders, meat powders, aqueous soluble polymers and combinations thereof. Virtually any finely divided hydrophilic particle may be used. Particles with a hydrophilic surface may be used regardless of the internal particle composition.

Smaller hydrophilic particles are preferred in creating products using the invention. Particles less than 500 microns may be used. But particles less than 50 microns are preferred, and particles less than 1 micron are most preferred. Nano sized particles, such as those used in the examples, function very well. This is probably due to the large available surface area available for interaction.

Examples of liquid polyhydric materials are glycerine, propylene glycol, dipropylene glycol, aqueous solutions of sorbitol, isomalt, lactitol, maltitol, aqueous solutions of sugars, starches, polyethylene glycol, aqueous soluble polymers, acids, dextrins, and combinations thereof. An aqueous solution of virtually any material may be used.

Polymers that are soluble in the hydrophobic phase and thus promote a higher liquid viscosity are useful adjuncts to the product, creating more stable structures and improving performance. Virtually any polymer that is soluble in the hydrophobic phase may be used, but preferred combinations include:
Ethylcellulose in flavor/fragrance at levels of about 0.1 to about 20% by weight of the structured material;
Hydroxypropyl cellulose in flavor/fragrance at levels of about 0.1 to about 20% by weight of the structured material;.
Ethylene vinyl acetate in flavor/fragrance at levels of about 0.1 to about 20% by weight of the structured material; Ethylcellulose in triglyceride oil at levels of about 0.1 to about 20% by weight of the structured material.

In addition, combinations of polymers are included. Additionally, hydrophobic phase materials may be combined in order to provide adequate solubilization of the selected polymer.

In addition to the foregoing components, various optional ingredients such as are conventionally used in the art, may be employed in the matrix of this invention. For example, colorants, pigments, hydrophobic particles, fillers, diluents, emulsifiers, preservatives, anti-oxidants, stabilizers, lubricants, and the like may be employed herein to enhance visual and/or functional characteristics.

The colorants of the present invention include, but are not limited to lakes, preparations containing lakes, oleoresins, pigments, and minerals. An example of a preparation containing lakes is Spectra Flecks™ (Sensient Technologies, St. Louis, Mo.).

Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Suitable diluents include calcium phosphate, calcium sulfate, carboxymethylcellulose calcium, cellulose, cellulose acetate, dextrates, dextrin, dextrose, fructose, glyceryl palmitostearate, hydrogenated vegetable oil, kaolin, lactitol, lactose, magnesium carbonate, magnesium oxide, maltitol, maltodextrin, maltose, microcrystalline cellulose, polymethacrylates, powdered cellulose, pregelatinized starch, silicified microcrystalline cellulose, sodium chloride, sorbitol, starch, sucrose, sugar, talc, hydrogenated vegetable oil, and mixtures thereof. Emulsifiers include mono and di-glycerol esters of fatty acids, modified starch, polyglycerol esters, and sorbitol esters.

These structured products may be produced effectively via batch or continuous processes, as long as there is sufficient mixing to disperse the solids in the hydrophobic liquid and to contact the liquid polyhydric material with the solids. An extrusion process which provides both intimate mixing, product formation, and the ability to post-treat particles is preferred when creating solid structures. A benefit of the invention is the fact that processing may take place at low temperatures, the only requirement being that the hydrophobic phase and the polyhydric material be liquids during the mixing.

The structured materials are useful for a variety of applications including:
Oral care applications, in which the structured materials can maintain discrete particles of flavor within an oral care product. Such oral care products include toothpaste, dental cream, gel or tooth powder, odontic, mouth washes, denture cleaning agents, pre- or post brushing rinse formulations, chewing gum, lozenges, candy and the like.
Baked products, in which the structured materials can help retain flavor during processing. Such baked products include bagels, biscuits, breads, cakes, cereal, cookies, crackers, cream puffs, doughnuts, empanadas, muffins, pancakes, pasta, pastries, pizza, sponge cakes and the like.
Cleaning products, in which the structured materials can delay release of fragrance, thus promoting increased fragrance deposition. Such cleaning products include detergents, soap, shampoo, hair conditioners, dishwashing compositions, scrubbing compounds, window cleaners and the like.

In order to demonstrate the invention, the following examples were conducted. All U.S. Patent and Patent applications referenced herein are hereby incorporated by reference as if set forth in their entirety.

Unless noted to the contrary all weights are weight percent. Upon review of the foregoing, numerous adaptations, modifications and alterations will occur to the reviewer. These adaptations, modifications, and alterations will all be within the spirit of the invention. Accordingly, reference should be made to the appended claims in order to ascertain the scope of the present invention.

### EXAMPLE 1

The following formulation was processed via extrusion at ambient temperature:

| | % by weight |
|---|---|
| Flavor | 70 |
| Silicon Dioxide - Aerosil 200 | 14.7 |
| Ethylcellulose Polymer | 4 |
| Glycerin | 11.29 |
| Color | 0.01 |

### EXAMPLE 2

Product from Example 1 was incorporated into a model toothpaste base. The particles survived the initial mixing process, retaining their visual identity in the base. When the toothpaste was evaluated, it was observed that the flavor onset from the particles was delayed several seconds compared to a control. Flavor from the particles continued to increase in intensity as brushing progressed. The particles were completely brushed out after 60 seconds. Flavor was still perceived 10 minutes post-brushing.

### EXAMPLE 3

Increase in Peppermint Flavor Viscosity

To demonstrate the increase in viscosity possible when using the invention, the following experiment was performed. Peppermint flavor (containing Peppermint oil, menthol, and other essential oils, but no solvent) was mixed by hand with Aerosil 200 (fumed silicon dioxide). Glycerine (99% purity) was added as indicated. Viscosity was measured (shear rate sweep) using a Haake AR 2000 Rheometer at ambient temperature (about 25° C.).

| | Con- | | | | |
|---|---|---|---|---|---|
| | trol | Sample 1 | Sample 2 | Sample 3 | Sample 4 |
| Peppermint | 14.00 | 14.00 | 14.00 | 14.00 | 14.00 |
| Flavor, grams | | | | | |
| Aerosil | | 1.00 | 1.00 | 2.00 | 2.00 |
| 200, grams | | | | | |
| Glycerine, | | | 0.72 | | 1.43 |
| grams | | | | | |
| Aerosil/ | | | 1.39 | | 1.40 |
| Glycerine ratio | | | | | |
| Comments | liquid | very | very | soft | semi- |
| | | thick | soft | solid | hard |
| | | liquid | solid | | solid |
| Viscosity | | | | | |
| (Pa · s) | | | | | |
| at specific | | | | | |
| shear rates | | | | | |
| 0.01/s | 0.058 | 54405 | 138700 | 479100 | 996250 |
| 0.02/s | 0.132 | 34730 | 79045 | 290200 | 618400 |
| 0.03/s | 0.083 | 21500 | 44965 | 177850 | 387250 |
| 0.04/s | 0.092 | 13300 | 27685 | 109900 | 245400 |
| 0.06/s | 0.065 | 8220 | 17100 | 67770 | 153750 |
| 0.10/s | 0.044 | 5073.5 | 10495 | 41720 | 82855 |

As can be seen, the addition of Aerosil increases the system viscosity by itself. Further addition of glycerine to the systems with dispersed Aerosil approximately doubles the viscosity, although the ratio of liquid to Aerosil increases significantly. This clearly shows the effect of the invention in creating structured systems.

1. A method for delivering an active ingredient to an ingestible composition comprising preparing a structured material comprising: providing a hydrophilic particle, wherein the hydrophilic particle is selected from the group consisting of hydrophilic silicas, silicates, dextrins, starches minerals, salts acids sodium bicarbonate, clays, sugars polyols, starches dextrins, dried flavors/fragrances, hydrocolloids, proteins, celluloses, hydroxypropyl methyl cellulose, methyl cellulose, flours of wheat, corn, potato, and rice, dried milk and dairy powders, spice, herb, and vegetable powders and meat powders less than 500 microns and wherein the amount of the hydrophilic particle used is from about 5% to about 70% by weight of the structured material; providing a hydrophobic active ingredient selected from the group consisting of flavors, fragrances, pharmaceutical agents, triglyceride oils, triglyceride fats, mineral oils and mixtures thereof; providing a polyhydric material selected from the group consisting of flavors, fragrances, pharmaceutical agents, triglyceride oils, triglyceride fats, mineral oils and mixtures thereof, wherein the amount of the polyhydric material used is from about 5% to about 20% by weight of the structured material; and further comprising providing a hydrophobically soluble polymer selected from the group consisting of hydroxypropyl cellulose, cellulose acetate butyrate, ethylene vinyl acetate, and ethylcellulose; admixing said hydrophilic particle, said active ingredient and said polyhydric material to form said structured material and providing said structured material into said ingestible composition.

2. (canceled)

3. (canceled)

4. A method as in claim 1, wherein the polyhydric material selected from the group consisting of glycerine, propylene glycol, dipropylene glycol, aqueous solutions of sorbitol, isomalt, lactitol, maltitol, aqueous solutions of sugars, starches, acids, salts, soluble polymers, and dextrins.

5. (canceled)

6. A method as in claim 1, wherein the hydrophilic particle is less than 50 microns.

7. A method as in claim 1, wherein the hydrophilic particle is less than 1 micron.

8. (canceled)

9. A method as in claim 1, wherein the amount of the polyhydric material used is from about 10% to about 15% by weight of the structured material.

10. A method as in claim 1, wherein the amount of the polyhydric material used is about 5% by weight of the structured material and wherein the viscosity measured using a Haake AR 2000 Rheometer and a shear rate sweep is from about 10,000 to about 140,000 Pa·s at about 25° C.

11. A method as in claim 1, wherein the amount of the polyhydric material used is about 8% by weight of the structured material and wherein the viscosity measured using a Haake AR 2000 Rheometer and a shear rate sweep is from about 80,000 to about 1,000,000 Pa·s at about 25° C.

12. (canceled)

13. A method as in claim 1, wherein the amount of the hydrophilic particle used is from about 15% to about 50% by weight of the structured material.

14. A method as in claim 1, wherein the amount of the hydrophilic particle used is from about 25% to about 40% by weight of the structured material.

15. A method as in claim 1, wherein the amount of the hydrophobic active ingredient used is from about 50% to about 90% by weight of the structured material.

16. A method as in claim 1, wherein the amount of the active ingredient used is from about 60% to about 70% by weight of the structured material.

17. A method as in claim 1, wherein the active ingredient is selected form the group consisting of triglyceride oils, triglyceride fats and mineral oils.

18. (canceled)

19. (canceled)

20. A method for delivering an active ingredient to a cleaning composition comprising preparing a structured material as in claim 1 and providing said structured material into said cleaning composition.

21. A An oral care product prepared by the method of claim 1, wherein the oral product is selected from the group consisting of toothpaste, dental cream, tooth powder, odontic, mouth washes, denture cleaning agents, pre-brushing rinses, post-brushing rinses, chewing gum and lozenges.
